(19) United States
(12) Patent Application Publication
(10) Pub. No.: US 2017/0204364 A1
(43) Pub. Date: Jul. 20, 2017

(54) METHOD FOR MODIFYING POLYSACCHARIDES BY GRAFTING POLYETHERAMINES, POLYSACCHARIDES THUS MODIFIED AND PREPARATIONS COMPRISING SAME AND HAVING HEAT-SENSITIVE RHEOLOGICAL PROPERTIES

(71) Applicant: CELENYS, Rouen (FR)

(72) Inventors: Zied SOUGUIR, Rouen (FR); Elise DEMANGE, Mont Saint Aignan (FR)

(21) Appl. No.: 15/324,934

(22) PCT Filed: Jul. 8, 2015
(86) PCT No.: PCT/FR2015/051883

§ 371 Date: Jan. 9, 2017

(30) Foreign Application Priority Data
Jul. 11, 2014 (FR) 1401564

(51) Int. Cl. C12N 5/00; B01D 61/14; C08G 81/00; C08B 37/00; C08G 65/337; C08B 37/08

(52) U.S. Cl. CPC C12N 5/0043; C08G 65/337; C08B 37/0072; C08G 81/00; C08B 37/0084; C08B 37/0018; C08B 37/0033; B01D 61/142; C08G 2650/50; C08G 2210/00; C08G 2650/58; C08G 2650/46; C08G 2650/38; C08G 2650/04; C12N 2533/74; C12N 2533/70; C12N 2533/80; B01D 2311/18; B01D 2315/16

(57) ABSTRACT

A modification of natural or synthetic polysaccharides by grafting polyetheramines, and the use of modified polysaccharides in the form of a hydrogel as a medium for cell culture. These hydrogel preparations may have heat-sensitive rheological properties that are interesting for their intracorporeal use in human and veterinary medicine, for cell culture and transport of biological samples.

### CROSS-REFERENCE TO RELATED APPLICATIONS

The present application is a National Stage Application of PCT International Application No. PCT/FR2015/051883 (filed on Jul. 8, 2015), under 35 U.S.C. §371, which claims priority to French Patent Application No. 1401564 (filed on Jul. 11, 2014), which are each hereby incorporated by reference in their respective entireties.

### TECHNICAL FIELD

The invention relates to organic chemistry, and more particularly to the chemistry of polysaccharides. It applies more precisely to the modification of natural or synthetic polysaccharides by grafting polyetheramines. It also relates to the use of these modified polysaccharides in the form of a hydrogel as a medium for cell culture. These hydrogel preparations can have heat-sensitive rheological properties that are interesting for their intracorporeal use in human and veterinary medicine, for cell culture and transport of biological samples (cells, excipients, biopsies, etc.).

### BACKGROUND

Hyaluronic acid is one of the most widespread polysaccharides in animal and human bodies. It can be manufactured on the industrial scale by fermentation from microorganisms (particularly Streptococcus equi). This product with a biological original is a biocompatible and biodegradable polysaccharide that can form hydrogels. This is why research was done to find intracorporeal applications for this product, particularly in orthopedics. Thus, intracorporeal use of hyaluronic acid (HA) hydrogels for the treatment of degraded or damaged cartilages is well known; the best-known process is viscosupplementation (i.e. the addition of HA to synovial liquid, or total replacement of the synovial liquid by HA). Intracorporeal use in dermatology has also been envisaged.

The idea of chemically modifying HA arose. This provides a means of generating hydrogels with new and special properties. This is described in detail in the publication entitled “Chemical modifications of hyaluronic acid for the synthesis of derivatives for a broad range of biomedical applications” by C. E. Schanté et al., published in the Carbohydrate Polymers journal, vol. 85, p. 469-489 (2011), in the PhD thesis by Zied Souguir (“Fonctionnalisation de polysaccharides et étude de leurs propriétés ‘pH dépendantes’”-Functionalization of polysaccharides and study of their ‘pH dependent’ properties)), Université de Rouen, 2006, and in the PhD thesis by Kristoffer Bergman “Hyaluronan Derivatives and Injectable Gels for Tissue Engineering”, Upsala 2008.

However, such a chemical modification must not induce direct or indirect toxicity (i.e. as a result of decomposition products during metabolization of the modified HA), and must not be prejudicial to biodegradability of the product.

Many patents deal with the chemical modification of HA and the use of modified HA for the treatment of joint pathologies. For example, EP 1 095 064 (Fidia) describes a number of HA derivatives. EP 2 457 574 A1 (Fidia Advanced Biopolymers) describes the preparation of biomaterials from derivatives of HA that are amides, and tests of their use in viscosupplementation. WO 2004/022603 (LG Life Sciences) describes HA polymers cross-linked with glycol based polymers, while KR 1007 37954 B1 (Korea University) describes an acrylated derivative of HA; these two documents envisage the intracorporeal use of the products obtained.

Grafting of hyaluronic acid by Jeffamine® type polyetheramines and by poly(N-isopropylacrylamides) (PNIPAM) is disclosed in the article entitled “Single step synthesis and characterization of thermoresponsive hyaluronan hydrogels” by M. D'Este et al., published in the Carbohydrate Polymers 90 journal (2012), p. 1378-1385.

Some of these modified (functionalized) HA hydrogels have heat-sensitive rheological properties. Intracorporeal applications for the controlled release of active constituents have been envisaged for these products, for example see the following publications: Mee Ryang Kim and Tae Gwan Park, “Temperature-responsive and degradable hyaluronic acid/Pluronic acid hydrogels for controlled release of human growth hormone”, Journal of Controlled Release, vol. 80, p. 69-77 (2002); T. R. Hoare and D. S. Kohane, “Hydrogels in drug delivery: Progress and challenges”, Polymer, vol. 49 (2008), p. 1993-2007; C. C. Chen et al., “Transdermal delivery of selegiline from alginate—Pluronic composite thermogels”, International J of Pharmaceutics, vol. 415 (2010), p. 25 119-128; S. Van Vlierberghe et al., “Biopolymer-Based Hydrogels As Scaffolds for Tissue Engineering Applications: A Review”, Biomacromolecules, 2011, 12, p. 1387-1408.

It is found that the choice of molecules grafted onto the polysaccharide is critical for the physicochemical characteristics of the product. More particularly, patent application EP 1 659 143 (Teijin) describes heat-sensitive hydrogels of hyaluronic acid and a secondary polyetheramine based on propylene oxide (Jeffamine® XTJ-507). The target application is the regeneration of cartilage. According to the information given in this document, the viscosity transition zone extends over an approximately 15° C. wide temperature range, which is too wide for an application in medicine.

Several publications suggest that the choice of polyetheramine can have an influence on the physicochemical properties of the product. For example, the article by G. Mocanu et al., “Multi-responsive carboxymethyl polysaccharide crosslinked hydrogels containing Jeffamine side-chains” published in Carbohydrate Polymers, vol. 89, p. 578-585 (2012) shows that, for a hydrogel containing a polysaccharide other than HA, there is a significant difference between the heat-sensitive properties of Jeffamines® M-600 and M-2005 (two products that are distinguished by their propylene oxide/ethylene oxide ratio and their molar masses). The “Single step synthesis and characterization of thermoresponsive hyaluronan hydrogels” publication by M. d'Este et al. (Carbohydrate Polymers, vol. 90, p. 1378-1385 (2012)) shows that for a HA Jeffamine® type hydrogel, Jeffamine® M2005 does not lead to significant heat-sensitivity, while the heat-sensitivity of the hydrogel with Jeffamine® M600 is perceptible but fairly low.

Heat-sensitive hydrogels of chitosan modified by acetylation or deacetylation are also known, see US 2009/0004276 (Mor Research Applications Ltd). It is also known that the modification of some polysaccharides provides a means of preparing hydrogels, some properties of which depend on the pH, see the thesis mentioned by Zied Souguir and the publication by G. Mocanu et al., “New anionic crosslinked multi-responsive pullulan hydrogels”, published in Carbohydrate Polymers, vol. 87, p. 1440-1446 (2012).

However in all these cases, the variation of the hydrogel properties as a function of the biological environment wherein it located in the case of intracorporeal use, and particularly the variation of its properties as a function of the temperature, is fairly small and fairly difficult to control during synthesis of the product.

There is obviously a need for a new approach to prepare a wider range of modified, biocompatible, non-toxic polysaccharides that can be used intracorporeally, and particularly that can be used for the transport and/or controlled release of active constituents and/or cells, that have heat-sensitive properties with a wider variation and that are easier to control during their synthesis.

### SUMMARY

According to the invention, the problem is solved by a new method of synthesis of modified polysaccharides by grafting polyetheramines. The applicant realized that the problem cannot be solved using available polyetheramines and that the first step would have to be the development of new polyetheramine molecules that can be grafted on a polysaccharide. As a result, new polysaccharides modified by grafting said polyetheramines could be prepared alongside new polysaccharide-based preparations that have particularly useful heat-sensitive rheological properties.

Thus, a first purpose of the invention is a method of modifying polysaccharides, wherein:

(a) a polysaccharide is made to react with a modified polyetheramine of the (X—R—C(O)NH)bR′ type (wherein R′ represents a polyether and b=1, 2 or 3 and X represents a halogen, preferably Cl or Br, R represents a possibly substituted alkyl group, or a possibly substituted aromatic group), in the presence of a base, preferably in the presence of water and isopropanol,

(b) the product obtained is purified at least partly in the presence of NaCl, by a membrane separation method, said purification being done at a pH between 9 and 13 (and preferably between 10 and 12);

(c) the product obtained in step (b) is at least partly purified by a membrane separation method, said purification being done after neutralization at pH between 6 and 8, (preferably between 6.5 and 7.5), possibly after freeze drying and washing of the freeze-dried product (preferably with ethanol).

R advantageously represents an alkyl group, possibly substituted (for example in C1, C2, C3, C4, C5, C6, C7, C8, C9, C10, C11 or C12) group, or an aromatic group (for example a possibly substituted phenyl group).

In one embodiment, said modified polyetheramine is a polyethermonoamine wherein the R′ group has the following structure:

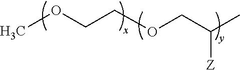

where Z is a hydrogen atom (in the case of ethylene oxide) or a methyl (in the case of propylene oxide), and wherein preferably x=1 to 3, Z═CH3 and y=7 to 11 (and preferably x=1 and y=9); x=17 to 21, Z═CH3 and y=2 to 5 (and preferably x=19 and y=3); x=5 to 8, Z═CH3 and y=25 to 32 (x=6 and y=29 preferred).

Advantageously, said polyetheramine R′ has a molar mass of between about 300 and about 3000, and even more preferably between about 500 and about 2500, and/or said polyetheramine with a [propylene oxide]/[ethylene oxide] molar ratio between 10/1 and 1/10.

In one advantageous embodiment of the method according to the invention, the first step is to activate at least one PS—COOH carboxylic function PS polysaccharide using a quaternary amine, and said modified polyetheramine is then added.

The method according to the invention can enable use of the modified polysaccharide in pharmaceutical or intracorporeal applications by including at least one purification step, and preferably all purification steps after neutralization, done at a temperature below 20° C., and preferably between 0° C. and 15° C., and even more preferably between 2° C. and 8° C.

In the polysaccharide modification method according to the invention, the product originating from step (c) can be freeze-dried, washed with ethanol and dried.

Advantageously, in preparation for its use in a hydrogel, said polysaccharide is selected from the group formed by neutral polysaccharides (and particularly pullulan and dextran), natural anionic polysaccharides (and particularly alginate, hyaluronic acid, xanthan gum, agar agar gum, pectins, heparin), synthetic anionic polysaccharides (and particularly carboxymethylcellulose, carboxymethylpullunan), natural cationic polysaccharides (and particularly chitosan), synthetic cationic polysaccharides (and particularly diethylaminoethylcellulose, diethylamoniethyldextran), amphiphilic polysaccharides, natural zwitterionic polysaccharides or polysaccharides obtained by chemical modification (and particularly carboxymethylchitosane), or mixes of these polysaccharides. Pullulan, xanthan, alginate and hyaluronic acid are preferred in particular.

Another purpose of the invention is a modified polysaccharide that can be obtained by the method according to the invention.

Yet another purpose of the invention is a hydrogel formed by at least one modified polysaccharide according to the invention and an aqueous liquid. Said aqueous liquid can include serum and/or a cell culture medium.

In one embodiment, the hydrogel according to the invention advantageously has heat-sensitive properties with a transition temperature of between 33 and 39° C. In another embodiment it has heat-sensitive rheological properties with a transition temperature equal to between 4 and 20° C.

Yet another purpose of the invention is the use of a hydrogel according to the invention in a cell culture medium or a cell transport medium.

Yet another purpose of the invention is the use of a hydrogel according to the invention for the preparation of a composition that will be used as a skin dressing, embolization agent, viscosupplementation agent, filling agent, post-surgical adhesion limitation agent, or a tissue regeneration agent.

Said modified polyetheramines of the (X—R—C(O)NH)bR′ type that are used in the polysaccharide modification method according to the invention do not exist in the state of the art and can be prepared by a new method for preparation of a modified (X—R—C(O)NH)bR′ type polyetheramine by the reaction of a polyetheramine (H2N)bR′ with a halide of a halogenated acyl X—R—C(O)X, wherein:

a polyetheramine (H2N)bR′ is procured (wherein R′ represents a polyether and b=1, 2 or 3) and a halogenated acyl halide X—R—C(O)X (wherein X represents a halogen, preferably Cl or Br, R represents a possibly substituted alkyl group, or a possibly substituted aromatic group),

the two reagents are mixed in the presence of a base, preferably Et3N and/or NaOH, and the mix is allowed to react at a temperature below 35° C., preferably below 25° C., even more preferably below 20° and optimally between 0° C. and 10° C., preferably in the absence of solvent (and preferably in the absence of DMF and THF).

The reaction takes place according to the following scheme:

X—R—C(O)X+(H2N)b-R′->(X—R—C(O)NH)bR′

wherein b is equal to 1, 2 or 3, wherein X represents a halide (preferably Cl or Br); R represents a possibly substituted alkyl group (for example a C6 group), or a possibly substituted aromatic group (for example a phenyl group) (but functionalization of the aromatic residue is not preferred); and R′ represents a polyether, preferably of the PPO or (PEO)x-co-(PPO)y type. The polyetheramine may be a polyethermonoamine (wherein b=1), or a polyetherdiamine (wherein b=2), or a polyethertriamine (wherein b=3). Primary amines are used in preference.

The reaction takes place in the presence of a base, preferably Et3N and/or NaOH that captures HX resulting from the reaction.

The reaction advantageously takes place at a temperature of below 20° C., preferably between 0° C. and 10° C.

The reaction medium can be washed with acidified water at the end of step (b). The modified polyetheramine obtained by this method can be preserved in the presence of an alcohol (preferably isopropanol).

In general, H2N—R′ polyethermonoamines, and particularly those with the following structure, are preferred for the fabrication of polysaccharides modified by grafting polyetheramines:

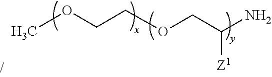

wherein Z1 is a hydrogen atom (in the case of ethylene oxide) or a methyl (in the case of propylene oxide), or an ethyl or propyl, and x and y indicate the chain length, knowing that x and y are integer numbers for a given molecule, but x and y represent average values for a given product in the state wherein it will be used (that may include molecules possibly with different lengths.

In one embodiment, the molar mass of polyetheramines that can be used can vary between about 300 and about 3000, and a range of between 500 and 2500 is preferred. The PO/EO molar ratio can vary within fairly wide limits, for example between 10/1 and 1/10.

But polyetherdiamines can also be used, and particularly those with the following structure:

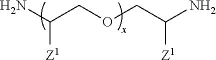

or the following structure:

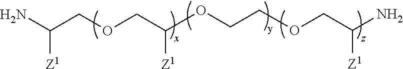

wherein Z1, x and y have the meanings mentioned above, and like x and y, z represents the chain length as described above.

Polyethertriamines can also be used, and particularly those with the following structure:

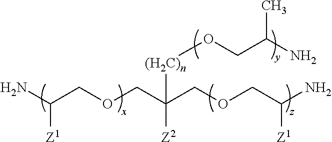

wherein Z1, x and y have the meanings mentioned above, and like x and y, z represents the chain length as described above. Z2 is hydrogen or an alkyl in C1 to C4, preferably methyl or ethyl. The number n may be between 0 and 12, and is preferably 0, 1 or 2.

Said polyetheramine advantageously has a molar mass of between about 300 and about 3000, and even more preferably between about 500 and about 2500. In the case wherein said polyetheramine is a mix of propylene oxide and ethylene oxide, said polyetheramine advantageously has a [propylene oxide]/[ethylene oxide] molar ratio equal to between 10/1 and 1/10.

In one advantageous embodiment and in particular for polyethermonoamines, x is chosen to be 1 to 7, Z═CH3 and y=5 to 15 (with x=1 to 3 and y=7 to 11 being preferred). In another advantageous embodiment, x is chosen to be 15 to 25, Z═CH3 and y=1 to 9 (with x=17 to 21 and y=2 to 5 being preferred). In yet another advantageous embodiment, x is chosen to be 3 to 11, Z═CH3 and y=21 to 35 (with x=5 to 8 and y=25 to 32 being preferred). These advantageous embodiments are produced in preparation for the use of modified polyetheramines resulting from the method according to the invention.

This new method of synthesizing modified polyetheramines as described above is another purpose of this invention.

Another purpose of the invention is modified polyetheramines of the (X—R—C(O)NH)bR′ type (where b=1, 2 or 3) that can be obtained by the method according to the invention. These polyetheramines are intermediate synthetic products that are useful for the preparation of modified polysaccharides, particularly as described above.

Thus, a final purpose of the invention is the use of a modified polyetheramine according to the invention to modify a polysaccharide.

### DRAWINGS

FIGS. 1 to 6, 8 and 9 illustrate different aspects of the invention, FIG. 7 illustrates the state of the art.

FIGS. 1 and 2 relate to a test of a modification of a Jeffamine® M2005 type polyetheramine by reaction with a halogenated acyl halide. The vertical axis represents the normalized intensity.

FIG. 1 shows the 1H NMR spectrum (in CDCl3) of the modified polyetheramine.

FIG. 2 shows the infrared spectra (FT-IR): curve (a) corresponds to the initial polyetheramine, curve (b) corresponds to the modified polyetheramine.

FIGS. 3 to 5 relate to a polysaccharide grafting test (in this case hyaluronic acid) with a Jeffamine® M2005 type polyetheramine that had previously been modified by reaction with a halogenated acyl halide (and that is the same as that in FIGS. 1 and 2).

FIG. 3 shows the 1H NMR spectrum (in D2O) of a hyaluronic acid (HA) grafted by modified polyetheramine.

FIGS. 4 and 5 show the variation of conservation modulus (elastic modulus) G′ (•) and the loss modulus (viscous modulus) G″(▾) of grafted HA as a function of the temperature: the concentration of grafted HA is 40 g/l in the RPMI culture medium (FIG. 4) or 20 g/l (FIG. 5). Note the sol-gel transition that is reversible for a temperature of less than 37° C. (about 29° C. for FIG. 4, about 34° C. for FIG. 5).

FIGS. 6 to 9 relate to tests described in more detail in the “Examples” section.

FIG. 6 shows the UV absorbance as a function of the temperature for a polysaccharide (in this case HA) grafted by a Jeffamine® M2005 type polyetheramine that had previously been modified by the action of 2-Bromo-2-methylpropionylbromide (Williamson reaction). The figure shows a measurement made on the reaction mix.

FIGS. 7 and 8 compare the variation of the conservation modulus (elastic modulus) G′ (curve A) and the loss modulus (viscous modulus) G″ (curve B) as a function of temperature for ungrafted hyaluronic acid (FIG. 7) and for grafted hyaluronic acid (grafting ratio: 2% molar) by a Jeffamine® M2005 type polyetheramine that had previously been modified by the action of 2-Bromo-2-methylpropionylbromide (esterification reaction) (FIG. 8). In both cases, the concentration of HA (grafted or not grafted) was 40 g/l in the RPMI culture medium.

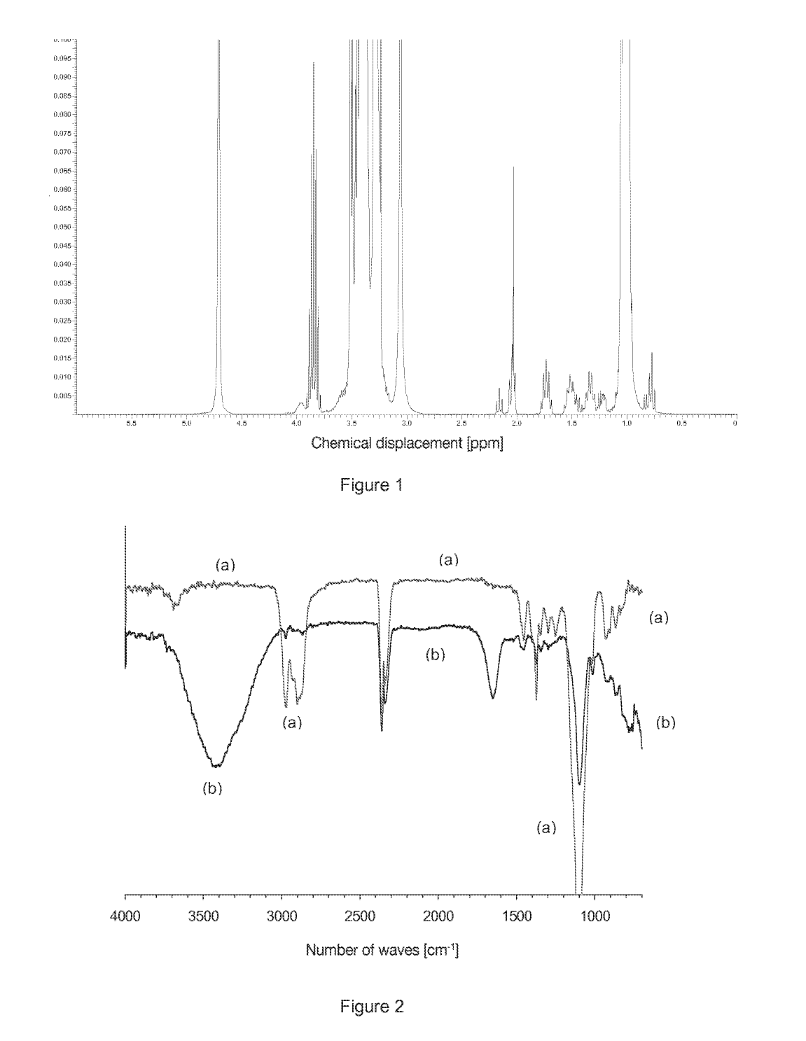

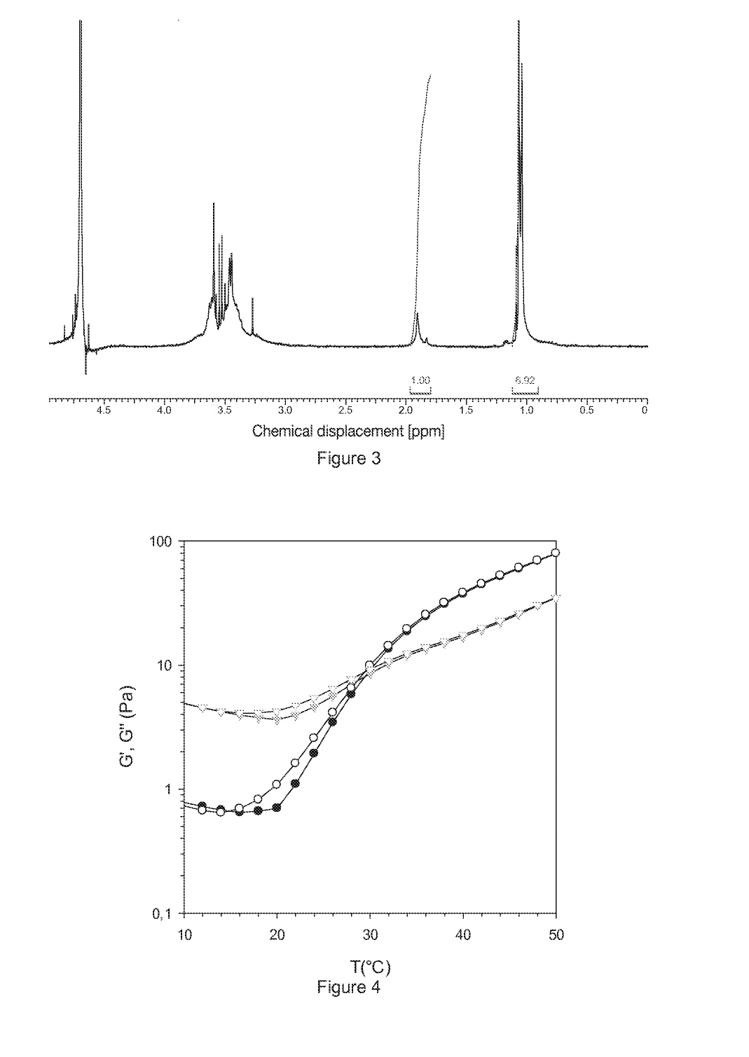

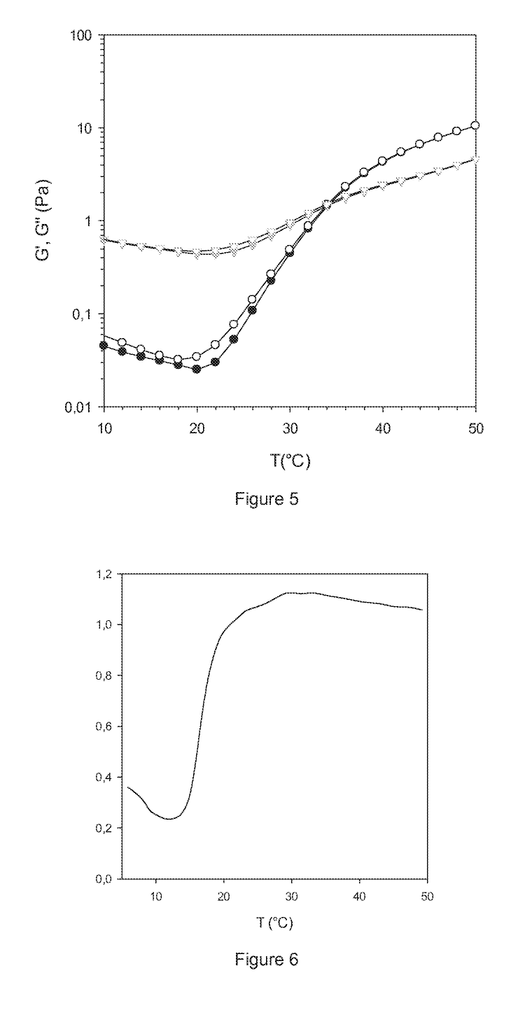

FIG. 9 shows DSC (Differential Scanning Calorimetry) curves for a sample of HA hydrogel grafted by a Jeffamine® M2005 type polyetheramine that had previously been modified by the action of 2-Bromo-2-methylpropionylbromide (esterification reaction); these hydrogels were formed with an RPMI type cell culture medium.
Curve A: HA hydrogel grafted by a modified Jeffamine®.
Curve B: Modified Jeffamine® (for comparison).
Curve C: Jeffamine® M2005 (for comparison).

### DESCRIPTION

Since modified polysaccharides according to the invention that give the best results are modified by grafting of polyetheramine derivatives that are not available off-the-shelf, we will start herein by describing a general method (section A) to obtain modified polyetheramines that could be grafted onto polysaccharides, and we will then describe two grafting methods (sections B and C) by esterification of polysaccharide to obtain polysaccharides with heat-sensitive rheological properties. These two methods give approximately the same products. And finally the use of these products is described (section D).

A) Modification of Polyetheramine by Reaction with a Halogenated Acyl Halide

This reaction takes place according to the following scheme:

X—R—C(O)X+(H2N)b-R′--->(X—R—C(O)NH)bR′

wherein b is equal to 1, 2 or 3, wherein X represents a halogen (preferably Cl or Br); R represents a possibly substituted alkyl group, (for example a group in C1, C2, C3, C4, C5, C6, C7, C8, C9, C10, C11 or C12), or an aromatic group (for example a possibly substituted phenyl group), (but functionalization of the aromatic group is not preferred); and R′ represents a polyether, preferably of the PPO or (PEO)x-co-(PPO)y type. The polyetheramine may be a polyethermonoamine (wherein b=1), or a polyetherdiamine (wherein b=2), or a polyethertriamine (wherein b=3). Primary amines are used in preference.

The reaction takes place in the presence of a base, preferably Et3N and/or NaOH that captures HX resulting from the reaction. The temperature is below 35° C., preferably below 25° C., and even more preferably below 20° C., and optimally between 0° C. and 10° C.; a temperature of about 4° C. is suitable.

Preferably the reaction of polyetheramine H2N—R′ with a halogenated acyl halide X—R—C(O)X is made without solvent. After the reaction, the mix is purified by a single washing with acidic water: this washing removes unreacted amine and HX acid (or its salt) resulting from the reaction.

The following diagram shows one advantageous embodiment of this reaction using a polyethermonoamine

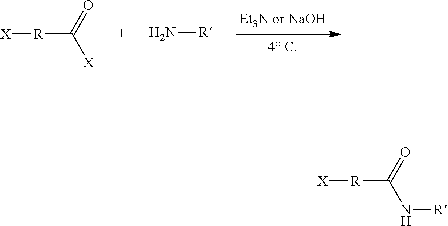

X: Halide (Cl, Br, etc.)

R: alkyl, aromatic, etc.

R′: PPO or (PEO)x-co-(PPO)y

A polyetherdiamine could also be used:

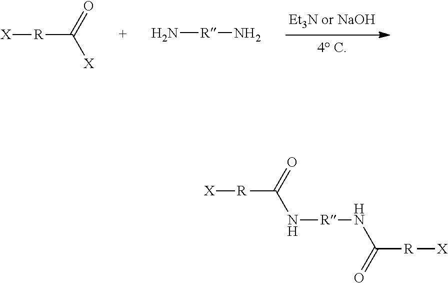

X: Halogen (Cl, Br, etc.)

R: alkyl, aromatic, etc.

R″: PPO or (PEO)x-co-(PPO)y

A polyethertriamine could also be used:

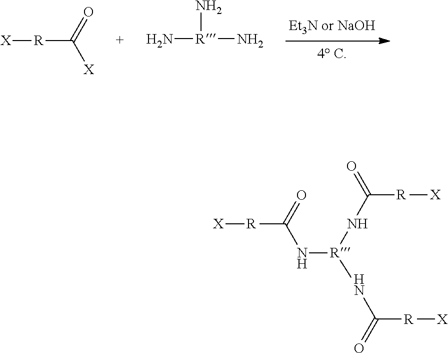

X: Halogen (Cl, Br, etc.)

R: alkyl, aromatic, etc.

R′″: PPO or (PEO)x-co-(PPO)y

In this description, PPO means polypropylene oxide and PEO means polyethylene oxide, and (PEO)x-co-(PPO)y is a copolymer between PO (propylene oxide) and EO (ethylene oxide). The Et3N abbreviation means triethylamine.

In particular, the polyetheramine H2N—R′ or R″(NH2)2 or R′″(NH2)3 used can be Jeffamine® brand products, and particularly Jeffamine® M600, Jeffamine® M2005, Jeffamine® M2070, Jeffamine® D2000 products, etc.). All these products are liquids, and the reaction can take place without solvent; as a general rule, the viscosity of the X—R—C(O)NHR′ product obtained is higher than the viscosity of the initial polyetheramine H2N—R′. The same comment is applicable to polyetherdiamines and polyethertriam ines.

The lack of a solvent (such as DMF, THF) for the reaction has several advantages; these solvents are expensive and toxic, and they have to be eliminated from the product afterwards because the product must not be contaminated with any molecules that could jeopardize its final use (that can be use in cell culture or even intracorporeal use).

The product can be conserved in isopropanol (in solution form), this solvent being chosen as a function of the subsequent use of the product. The product can be characterized by NMR and Infrared spectroscopies to demonstrate its identity and its purity.

This method according to the invention has many advantages. It provides access to a wide spectrum of modified polyetheramines with good purity. It can be done without organic solvents (such as DMF, THF) that could possibly hinder the use of polyetheramines according to the invention, even in trace quantities, for the preparation of modified polysaccharides for pharmaceutical or intracorporeal use.

We will now describe the use of modified polyetheramines according to the invention to demonstrate the industrial application of this new class of compounds as intermediate synthesis products, and in particular so as to obtain modified polysaccharides with a variety of useful physical, physicochemical and chemical properties, for use in pharmacies, cellar biology or in medicine.

B) Modification of Polysaccharides by Williamson's Reaction

This method is aimed at grafting the modified polyetheramine that can be obtained by the method according to the invention, and particularly according to step 1 described above, onto a polysaccharide, in particular to obtain polysaccharides with heat-sensitive rheological properties. An alternative method is presented below (method C).

The reaction includes grafting of an (X—R—C(O)NH)bR′ type modified polyetheramine (for example a modified polyethermonamine of the X—R—C(O)NHR′ type, or a modified polyetherdiamine of the (X—R—C(O)NH)2R′ type, or a modified polyethertriamine of the (X—R—C(O)NH)3R′ type, on a —OH group of a polysaccharide (PS—OH), which leads to a modified polysaccharide of the (PS—O—R—C(O)NH)bR′ type, (in the case of a modified polyethermonamine, the modified polysaccharide can be of (PS—O—R—C(O)NHR′ type, in the case of a modified polyetherdiamine, the modified polysaccharide can be of (PS—O—R—C(O)NH)2R′ type, and in the case of a modified polyethertriamine, the modified polysaccharide can be of (PS—O—R—C(O)NH)3R′ type, wherein the oxygen atom that forms the grafting point between the polysaccharide and the graft originates from the OH group of the polysaccharide

In this reaction scheme, the X, R and R′ symbols have the same meanings as those described above with reference to method A.

The reaction preferably takes place in a mix of water and isopropanol, making direct use of the product from method A described above.

The following scheme shows one advantageous embodiment of this reaction:

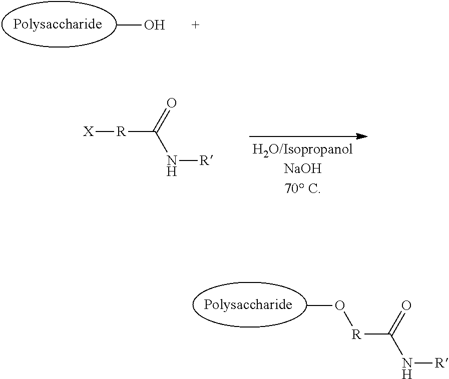

X: Halogen (Cl, Br, etc.)

R: alkyl, aromatic, etc.

R′: PPO or (PEO)x-co-(PPO)y

Physicochemical analyses made in a cell culture medium (RPMI) by rheological measurements, and measurements of the conservation modulus (G′) and loss modulus (G″) as a function of the temperature for two concentrations (40 and 20 g/l) show the reversible sol gel transition for temperatures of less than 37° C.

C) Modification of Polysaccharides by Esterification

This method is aimed at grafting the modified polyetheramine that can be obtained by the method according to the invention, obtained for example according to step 1 described above, onto a polysaccharide, in particular to obtain polysaccharides with heat-sensitive rheological properties. It is an alternative to method B presented above.

The reaction includes activation of the carboxylic function PS—COOH of a polysaccharide PS using a quaternary amide, and preferably a tetrabutylamine (TBA) leading to a PS—COO— function, followed by grafting a modified polyetheramine of the (X—R—C(O)NH)bR′ type on said —COO— group of the polysaccharide, which leads to a (PS—COO—R—C(O)NH)bR′ type of modified polysaccharide, wherein the oxygen atom that forms the grafting point between the polysaccharide and the graft originates from the COOH group of the polysaccharide.

In this reaction scheme, the X, R and R′ symbols have the same meanings as those described above with reference to method A.

The reaction preferably takes place in a mix of water and isopropanol, making direct use of the product from method A described above. A temperature higher than 25° C. is preferred, preferably between 40° C. and 95° C., and even more preferably between 55° C. and 90° C., a temperature of about 70° C. is quite suitable.

The following scheme shows one advantageous embodiment of this reaction:

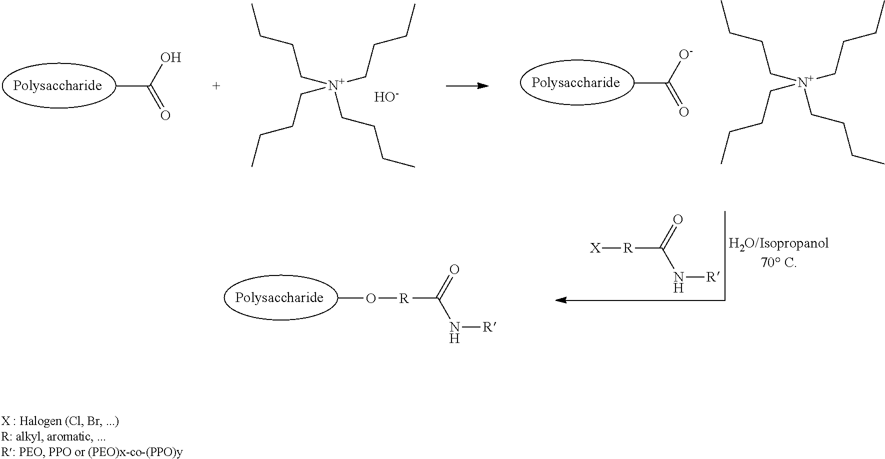

Comments Common to Methods B and C

a) Purification

The reaction product derived using method B or C must be purified if it is to be used for intracorporeal use. This purification is advantageously made in the presence of NaCl and in at least two steps distinguished by their pH value.

A first purification step is done at a pH between 9 and 13 (preferably between 10 and 12, and even more preferably about 11, and preferably at least partly (and possibly entirely) using a membrane separation method such as diafiltration. This is the pH value at which unreacted polyetheramine molecules (i.e. molecules that have not been grafted onto polysaccharide) are eliminated, probably by neutralization of quaternary ammonium functions of polyetheramine and elimination of ionic bonds between ammonium in the polyetheramine and polysaccharide carboxylate; unreacted polyetheramine usually has cellular toxicity that is a problem for subsequent use of hydrogel in cell culture, and would always be unacceptable for intracorporeal use of the hydrogel.

A second purification step is performed after neutralization, preferably at a pH of about 7, which is usually the pH at which the hydrogel will be used later, either for cell culture or for intracorporeal applications. This second purification step can also be made either partially or entirely, by a membrane separation method such as diafiltration, or by another appropriate technique. This second step at neutral pH can be done after freeze-drying (the powder subsequently being washed with ethanol to remove remaining polyetheramine groups and other by-products).

Membrane separation can be done in a known manner, for example with membranes with an MWCO (Molecular Weight Cut-Off) value equal to about 10 kDa to 30 kDa, for example between 12 kDa to 14 kDa in sausage mode. Dialysis can be done against water and/or against a mix of water and ethanol (for example with a water to ethanol ratio by volume equal to 2/3-1/3). A reduction of the pH during dialysis is observed.

According to one very advantageous aspect of the invention, at least one purification step (and preferably at least all purification steps before neutralization, and even more preferably also at least one (and preferably all) purification steps after neutralization) is (are) done at a temperature of less than 20° C., preferably between 0° C. and 15° C., and even more preferably between 2° C. and 8° C., particularly at a temperature of about 4° C.

The applicant has observed that at ambient temperature, the reaction mix is cloudy and tends to form lumps at ambient temperature that hinder purification; it is necessary to obtain a pure product for any intracorporeal use of the hydrogel. On the other hand, unlike many polysaccharides according to the state of the art, the product according to the invention purified at low temperature as mentioned above, is translucid after gelling.

The applicant tends to believe (without being limited to this idea) that unmodified (free) polyetheramine participates in the formation of said aggregates wherein it could be trapped, since the aggregates disappear after sufficient purification of the hydrogel.

The applicant has found that apart from the toxicity of residual polyetheramine (unreacted, free), there is another reason to maximize purification of modified polysaccharide by grafting a polyetheramine; the presence of free polyetheramine in hydrogels that show a variation of viscosity as a function of the temperature leads to a lower viscosity gradient spread over a wider temperature range then a purified hydrogel.

The purified product is frozen (for example at −20° C.) and freeze-dried and then washed with ethanol (for example twice) and dried (preferably at 40° C. under a vacuum).

The final product is in the form of a dry powder. It can be transformed into a hydrogel by dispersing it in a required quantity of an aqueous medium. Said aqueous medium may be a cell culture. For example, culture media known as RPMI (Roswell Park Memorial Institute) can be used. The aqueous medium may include additives such as growth factors and/or pharmaceutically active constituents (such as antibiotics) and serum.

b) Usable Polysaccharides

Different types of polysaccharides can be used in the framework of this invention, to be modified by grafting according to method B or method C. These polysaccharides may belong to groups of neutral polysaccharides (for example pullulan, dextran), natural anionic polysaccharides (for example alginate, hyaluronic acid, xanthan gum, agar agar gum, pectins, heparin), synthetic anionic polysaccharides (for example carboxymethylcellulose, carboxymethylpullunan), natural cationic polysaccharides (particularly chitosan), synthetic cationic polysaccharides (for example diethylaminoethylcellulose, diethylamoniethyldextran), amphiphilic polysaccharides, natural zwitterionic polysaccharides or those obtained by chemical modification (for example carboxymethylchitosane).

Pullulan, xanthan, alginate, hyaluronic acid (HA) type polysaccharides are particularly preferred to prepare grafted polysaccharides with heat-sensitive rheological properties. Cell culture tests at different concentrations demonstrate that these products are not toxic and cell proliferation in these systems.

These polysaccharides are also biocompatible and biodegradable.

For example, hyaluronic acid (HA) with its well-known biocompatibility can be used. We can use an HA with bacterial origin (Streptococcus equi) available off-the-shelf with a molar mass number typically varying from 10³ to more than 10⁶ g/mol (determined by steric exclusion chromatography, multi-angle diffusion of light and refractometry).

c) Grafting Ratio

The method according to the invention can obtain high grafting ratios of up to 20% molar. The applicant is not aware of any known method that would allow to obtain such high grafting ratios. For the use of products as polysaccharides with heat-sensitive rheological properties, it is preferred to use a grafting ratio of between 5% and 20% molar, preferably between 5% and 15%, and even more preferably between 10% and 15%. In some grafted systems with grafting ratios of more than 15 to 20%, the heat-sensitivity effect of rheological properties tends to decrease.

d) Choice of Polyetheramines

Polyetheramines preferred for the purposes of this invention (this “Choice of polyetheramines” section applies to methods A, B and C) are copolymers of the polyethers type composed of propylene oxide (PO) and ethylene oxide (EO). The presence of these propylene oxides makes the macromolecule hydrophobic and heat-sensitive, creating a precipitation in aqueous solution as a function of the temperature. The temperature of this transition depends among other factors on the relative PO/EO quantity.

Polyetheramines used for this invention are preferably primary amines.

In general, polyethermonoamines are preferred (particularly for methods B and C) and particularly those with the following structure:

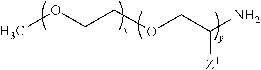

wherein Z1 is a hydrogen atom (in the case of ethylene oxide) or a methyl (in the case of propylene oxide), and x and y indicate the chain length, knowing that x and y are integer numbers for a given molecule, but x and y represent average values for a given product in the state wherein it will be used (that may include molecules possibly with different lengths.

The molar mass of polyetheramines that can be used can vary between about 300 and about 3000, and a range of between 500 and 2500 is preferred. The PO/EO molar ratio can vary within fairly wide limits, for example between 10/1 and 1/10.

For example, the following polyethermonoamines can be used:

x=1 to 3, Z1═CH3 and y=7 to 11 (with x=1 and y=9 preferred); x=17 to 21, Z1═CH3 and y=2 to 5 (with x=19 and y=3 preferred); x=5 to 8, Z1═CH3 and y=25 to 32 (with x=6 and y=29 preferred).

But polyetherdiamines can also be used, and particularly those with the following structure:

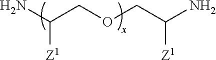

or the following structure

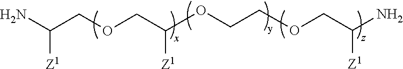

wherein Z1, x and y have the meanings mentioned above, and like x and y, z represents the chain length as described above.

Polyethertriamines can also be used, and particularly those with the following structure:

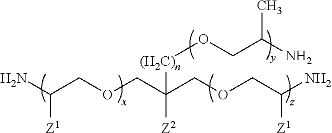

wherein Z1, x and y have the meanings mentioned above, and like x and y, z represents the chain length as described above. Z2 is hydrogen or an alkyl in C1 to C4, preferably methyl or ethyl. The number n may be between 0 and 12, and is preferably 0, 1 or 2.

For example it is possible to use polyethermonoamines marketed as the Jeffamine® brand (series M), or polyetherdiamines marketed as the Jeffamine® brand (series D, ED), or polyethertriamines marketed as the Jeffamine® brand (series T), by the Huntsman company.

D) Use of Polysaccharide Hydroqels Modified by Grafting According to the Invention

Polysaccharide hydrogels modified by grafting according to the invention can be used in biology and in medicine, either extracorporeally or intracorporeally. These hydrogels can be prepared with water or aqueous liquids such as buffered aqueous solutions, physiological serum, standard or special cell culture media.

These uses in biology and in medicine are possible due to the possibility of very efficiently purifying modified polysaccharides according to the invention, in order to eliminate all toxic residue. Some uses, particularly intracorporeal uses, are also possible due to the rheology of modified polysaccharides according to the invention (orthopedics, cosmetology (for example infilling of wrinkles, dermatology).

Extracorporeal uses include use as a cell culture medium, particularly for human and animal cells, or use in a cell culture medium composition, particularly for animal or human cells. In particular, these hydrogels can also be used in microfluidics systems. They also include use as a cell storage and/or transport medium (or in a medium composition), or a biopsies or explants medium, particularly for animal or human cells. Hydrogels according to the invention have a three-dimensional network that embeds cells to be cultivated under conditions conducive to their growth and multiplication.

Intracorporeal uses include use as a skin dressing, embolization agent, viscosupplementation agent, filler, agent limiting post-surgical adhesion, as a tissue regeneration agent, or in the composition of such agents.

In particular, these applications can take advantage of the heat-sensitive properties of the hydrogel according to the invention.

We will now describe a typical use of a modified polysaccharide hydrogel with heat-sensitive rheological properties according to the invention. The hydrogel is solubilized in the cell culture medium so that it can be used as a three-dimensional cell culture medium, and the cells are deposited in the heat-sensitive gel at ambient temperature. When the temperature of the system is increased (up to 37° C., incubator temperature), cells are sequestered inside the hydrogel. One important heat-sensitive characteristic is optical transparency when the system is in gel form so that a microscopic analysis can be made. Cells can then develop in suspension inside the system and proliferate in this system. Cells can be recovered and analyzed at the time of the next transition to ambient temperature.

Hydrogels with heat-sensitive rheological properties can also be used to transport cells (stem cells, primary cells and ligneous cells) or for taking samples (such as biopsies) at a temperature of about 37° C. These valuable samples are subjected to shocks and shaking during transport, and are often damaged on arrival. Hydrogel with heat-sensitive rheological properties can then limit the impact of such shaking due to handling of dispatches by sequestering cells or samples (such as biopsies) inside the hydrogel. Once the addressee has received the sample, all that is necessary is to return to ambient temperature to liquefy the medium and thus easily recover the cells or samples contained in it.

Hydrogel with heat-sensitive rheological properties can also be used in regenerative medicine, for example for regeneration of a cartilage. At the present time, one of the main techniques used for cartilage regeneration is micro fracture. The physician punches the bone subjacent to the cartilage on a patient suffering from a grade III or IV lesion. These punch marks lead to the effusion of blood containing stem cells. These stem cells have the ability to regenerate the cartilage. However, there is a problem with this technique in that the cells do not always remain on the injured site and disperse. The injection of a hydrogel with heat-sensitive rheological properties filled with blood containing stem cells (or another biological medium enriched with stem cells or containing stem cells) during the microfracture could localize cells on the site of the lesion and facilitate regeneration of the cartilage.

E) Advantages of the Invention

The use of modified polyetheramines according to the invention as a graft can result in grafted polysaccharides with new physicochemical characteristics, and in particular with a viscosity that depends on the temperature. Higher grafting ratios can be obtained through the use of polyetheramines according to the invention.

Purification of hydrogels at low temperature can give purer, non-toxic hydrogels, without any free polyetheramine. The absence of free polyetheramine also accentuates the variation of the viscosity as a function of the temperature and narrows the temperature wherein the viscosity transition takes place.

Hydrogels according to the invention may have heat-sensitive rheological properties, passing from a liquid state to a state with a higher viscosity wherein they form a three-dimensional nanostructure; they can hold cells in this state. They are optically transparent and thus enable optical observation of said cells.

### EXAMPLES

The following examples are given solely for illustration, so that a person skilled in the art can carry out the invention. They do not limit the scope of the invention.

### I. Examples Applicable to the Synthesis of Polyetheramine Derivatives

### Example 1

Modification of a polyethermonoamine with x=6, Z═CH3 and y=29 by chloroacetyl chloride in the presence of triethylamine (TEA)

100 g (50 mmol) of polyethermonoamine (x=6, Z═CH3 and y=29, a product available as the Jeffamine® M2005 brand with molecular mass equal to about 2000 g/mol), and 6.7 ml (50 mmol) of triethylamine (TEA,) were added into a reactor with a volume of 250 ml. The mix was then placed in an ice bath at 4° C., and 3.96 ml (50 mmol) of chloroacetyl chloride was added drop by drop with strong magnetic stirring. The reaction mix was kept for 2 hours. 100 ml of 2-propanol was added after 2 hours. The mix was then transferred into a separation funnel, and 200 ml of water with acid pH was added (pH=3). After the phase separation, the organic phase containing modified Jeffamine® M2005 was recovered.

### Example 2

Modification of a polyethermonoamine with x=6, Z═CH3 and y=29 by chloroacetyl chloride in the presence of soda (NaOH)

100 g (50 mmol) of polyethermonoamine (x=6, Z═CH3 and y=29, a product available as the Jeffamine® M2005 brand, and 2 ml of a 50 mmol solution of soda (NaOH), were added into a reactor with a volume of 250 ml. The mix was then placed in an ice bath at 4° C., and 3.96 ml (50 mmol) of chloroacetyl chloride was added drop by drop with strong magnetic stirring. The reaction mix was kept for 2 hours. 100 ml of 2-propanol was added after 2 hours. The mix was then transferred into a separation funnel, and 200 ml of water with acid pH was added (pH=3). After the phase separation, the organic phase containing modified Jeffamine® M2005 was recovered.

### Example 3

Modification of a polyethermonoamine with x=1, Z═CH3, and y=9 by 2-Bromo-2-methylpropionyl bromide (BIBB) in the presence of triethylamine (TEA)

100 g (166 mmol) of polyethermonoamine (x=1, Z═CH3 and y=9, a product available as the Jeffamine® M600 or XTJ-505 brand with molecular mass equal to about 600 g/mol) and 22.5 ml (166 mmol) of triethylamine (TEA) were added into a reactor with a volume of 250 ml. The mix was then placed in an ice bath at 4° C., and 20.5 ml (166 mmol) of chloroethyl chloride was added drop by drop with strong magnetic stirring. The mix was kept for 2 hours. 100 ml of 2-propanol was added to the reaction mix after 2 hours. The mix was then transferred into a separation funnel, and 200 ml of water with acid pH was added (pH=3). After the phase separation, the organic phase containing modified Jeffamine® M600 was recovered.

### Additional Example

The following modified polyethermonoamines were made according to one of the procedures described above:

Basic polyethermonoamine:

(a) Z═CH3, x=6, y=29 (off-the-shelf as Jeffamine® M2005 brand)

(b) Z═CH3, x=1, y=9 (off-the-shelf as Jeffamine® M600 brand)

(c) Z═H (for EO) or CH3 (for PO), x=6, y=35 (Jeffamine® M2070)

Each of these three polyethermonoamines was modified by the reaction with:

(a1, b1, c1): chlorylacetyl chloride

(a2, b2, c2): α-bromoisobutyryl bromide

(c1, c2, c3): 6-bromohexanoyl chloride.

It has also been checked that these syntheses can be made with polyetherdiamines and polyethertriamines.

### II. Examples Applicable to the Modification of Polysaccharides by Polyetheramine Derivatives

### Example 4

Grafting of Jeffamine® M2005 modified by 2-Bromo-2-methylpropionyl bromide on hyaluronic acid (HA) by the Williamson reaction.

1 g (2.5 mmol) of HA was solubilized in 100 ml of water in a reactor, with mechanical stirring. The solution was heated to 70° C. 0.8 g of NaOH (0.2 mol) was then dissolved in 5 ml of water and this soda solution was then added to the medium with 0.5 g of sodium iodide. After 15 minutes, 11 ml of modified Jeffamine® M2005 solution was then added into the isopropanol. The reaction mix was heated to 70° C. for 4 h, and then 2 g of NaCl was added and the pH was then adjusted to 11 by the addition of HCl (1M). The mix was kept at 4° C. for 12 hours.

Purification was obtained by diafiltration at 4° C. Absorbance measurements of the reaction mix by UV as a function of temperature (FIG. 6) demonstrate an increase in absorbance that indicates the presence of a cloudy phase due to the formation of aggregates. This observation implies that the mix cannot be purified at ambient temperature.

The diafiltration system was set to 4° C. to purify reaction mixes efficiently. Purification was done in 3 steps. Diafiltration was started at pH=11, passing the volume of the initial solution 3 times. The solution was then neutralized at pH=7, passing the volume of the solution 4 times. Diafiltration was stopped after the conductivity was checked. The sample was frozen and then freeze-dried. The lyophilisate was washed with absolute ethanol and finally dried under a vacuum.

### Example 5

Grafting of Jeffamine® M2005 modified by 2-Bromo-2-methylpropionyl bromide on hyaluronic acid (HA) by the esterification reaction.

1 g (2.5 mmol) of HA was solubilized in 100 ml of water in a reactor, with mechanical stirring. The pH of the solution was then adjusted by the addition of HCl (1M). The mix is dialyzed against pure water for 24 hours. After dialysis, the solution is neutralized to pH=7 by the addition of tetrabutylammonium hydroxide (TBAOH). For the grafting reaction, the solution of neutralized HA was heated to 70° C. 11 ml of the modified Jeffamine® M2005 solution was then added into isopropanol. The reaction mix was then heated to 70° C. for 12 h. 2 g of NaCl was then added and the mix was conserved at 4° C. for 12 h. The purification method used was the same as that for example 4.

### Example 6: Rheological Properties of Polysaccharide Hydrogels as a Function of the Temperature

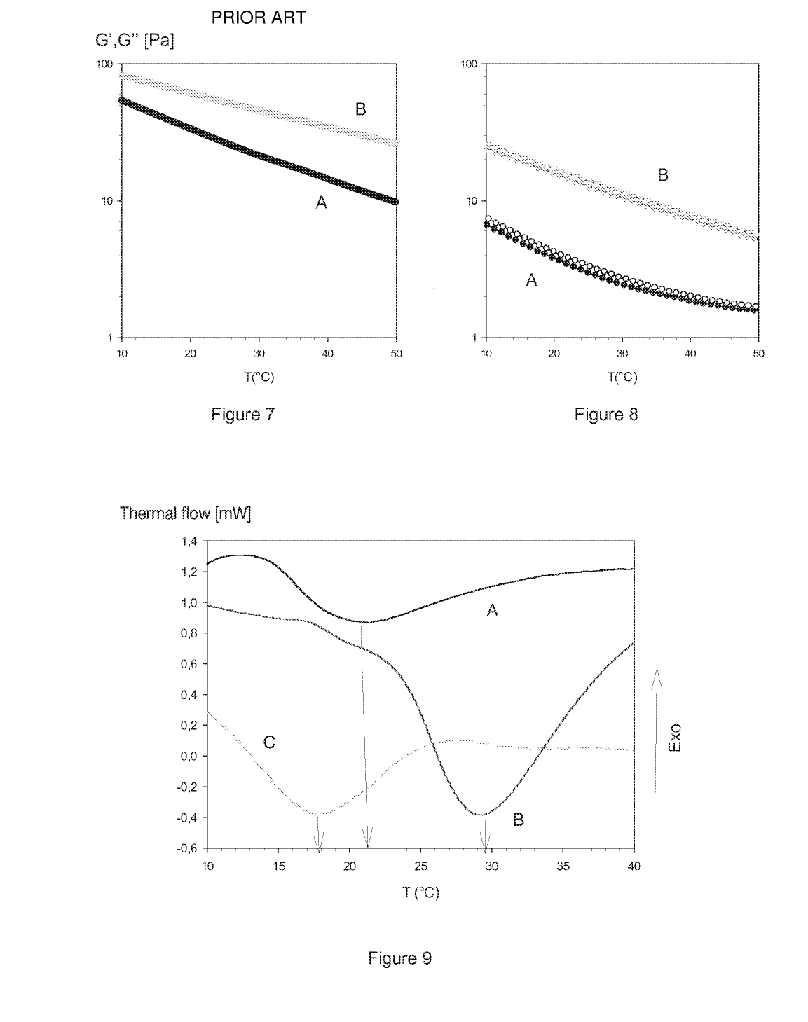

The elastic modulus G′ and the viscous modulus G″ of different polysaccharide hydrogels as a function of the temperature were measured, at a concentration of 40 g/l in the RPMI culture medium. The results are shown on FIGS. 7 and 8.

For unmodified HA (FIG. 7), these measurements show that the system does not have any heat-sensitive transition (no gelling) and all that is observed is simply a reduction in the modulus values as a function of the temperature, which is a classical phenomenon for polymers.

For modified HA using the method according to the invention (BIBB-Jeffamine® M-2005 with a grafting ratio of 2%) these measurements show (FIG. 8) that there is no heat-sensitive transition with this system (no gelling), and all that is observed is a reduction of the modulus values as a function of the temperature.

### Example 7: Reversible Sol-Gel Transition in a Hydrogel for Cell Culture

An HA hydrogel grafted by a modified Jeffamine® type polyetheramine according to the invention was prepared with a culture medium known as RPMI (Roswell Park Memorial Institute Medium). A clear liquid was obtained at 20° C. that becomes a gel at 37° C., but remains clear and translucid. Solidification of the liquid is reversible.

### Example 8: Reversible Sol-Gel Transition of a Hydrogel for Cell Culture

An HA hydrogel grafted by a modified Jeffamine® 2005 type polyetheramine according to the invention was prepared using a halide acid (BIBB-Jeffamine® M-2005 with a grafting ratio of 10%) in a DMEM (“Dulbecco/Vogt modified Eagle's Minimal Essential Medium”) type medium buffered by 4-(2-hydroxyethyl)-1-piperazine ethane sulfonic (HEPES) acid; this cell culture medium is known as HDMEM or hDMEM.

A DSC (Differential Scanning Calorimetry) thermogram was made at a rate of 2° C.min-1. This technique measures enthalpy variations as a function of the temperature. The recorded thermogram is shown on FIG. 9, curve A. The same thermogram for Jeffamine® modified by a halide acid (curve B) and for Jeffamine® M2005 (curve C) were also recorded for comparison.

An endothermal phenomenon is observed as the temperature rises from 10° C. to 40° C.; this is probably due to the formation of hydrophobic associations.

Note that not all the peaks are located at the same temperature. The comparison of peak temperatures shows that the modification of polyetheramine by a halide acid reduces its transition temperature (formation of hydrophobic associations) from about 29° C. to about 18° C. It also shows that after grafting this modified polyetheramine onto an HA type polysaccharide, the transition temperature of the system is closer to the transition temperature of modified polyetheramine that to that of unmodified polyetheramine.

The behavior is reversible with an exothermal phenomenon during the cooling phase from 40° C. to 10° C. (not shown on the graph).

### Example 9: Grafting of Modified Polyethermonoamines onto Miscellaneous Polysaccharides

The following modified polysaccharides were prepared using the approaches described in the previous examples:

(i) with the basic polyethermonoamine corresponding to Z═CH3, x=6, y=29 (available off-the-shelf as the Jeffamine® M2005 brand) modified by the reaction with a-bromoisobutyryl bromide:
Polysaccharide=hyaluronic acid (HA) with a grafting ratio of between 2% and 21% (molar).
Polysaccharide=alginate with a grafting ratio of 1%
Polysaccharide=pullulan with a grafting ratio of 4%
Polysaccharide=xanthan with a grafting ratio of 2%

(ii) with the basic polyethermonoamine corresponding to Z═CH3, x=6, y=29 (available off-the-shelf as the Jeffamine® M2005 brand) modified by the reaction with 6-bromohexanoyl chloride:
Polysaccharide=hyaluronic acid (HA) with a grafting ratio of between 2% and 10% (molar)
Polysaccharide=alginate with a grafting ratio of 5%
Polysaccharide=diethylaminoethylpullulan (DEAE-pullulan) with a grafting ratio of 10%

(iii) with the basic polyethermonoamine corresponding to Z═CH3, x=1, y=9 (available off-the-shelf as the Jeffamine® M600 brand) modified by the reaction with 6-bromohexanoyl chloride:
Polysaccharide=hyaluronic acid (HA) with a grafting ratio of 1% (molar)

(iv) with basic polyethermonoamine corresponding to Z═H (for EO) or CH3 (for PO), x=6 y=35 (available off-the-shelf as the Jeffamine® M2070 brand) modified by the reaction with a-bromoisobutyryl bromide:
Polysaccharide=hyaluronic acid (HA) with a grafting ratio of 18% (molar)

The best results for use as a hydrogel with heat-sensitive rheological properties with a transition at about a normal body temperature (about 37°) were achieved with grafted HA at 3 to 10% molar with the basic polyethermonoamine corresponding to Z═H (for EO) or CH₃ (for PO), x=6, y=35 (off-the shelf as the Jeffamine® M2070 brand) modified by the reaction with 6-bromohexanoyl chloride.

1-20. (canceled)

21. A method for modifying polysaccharides, the method comprising:
(a) reacting a polysaccharide with a modified polyetheramine of the (X—R—C(O)NH)_{b}R′ type, wherein R′ is a polyether, b=1, 2, or 3, X is a halogen, R is an alkyl group or an aromatic group, in the presence of water and isopropanol;
(b) purifying, at least partially, the product obtained (a) in the presence of NaCl, by a membrane separation method, wherein the purification is carried out at a pH between 10 and 12;
(c) purifying, at least partially, the product obtained from (b) by a membrane separation method, wherein the purification is carried out after neutralization at a pH of between 6.5 and 7.5.

22. The method of claim 21, wherein:
the alkyl group is a C₁, C₂, C₃, C₄, C₅, C₆, C₇, C₈, C₉, C₁₀C₁₁ or C₁₂ group, and
the aromatic group is a phenyl group.

23. The method of claim 21, wherein the R′ has a following structure: 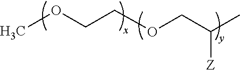
where Z is a hydrogen atom (in a case of ethylene oxide), or a methyl (in a case of propylene oxide),
x=Z ═CH₃, and y=9, or
x=19, Z═CH₃, and y=3, or
x=Z ═CH₃, and y=29,
wherein:
said polyetheramine has a molar mass of between about 500 and about 2500, and/or
said polyetheramine presents a [propylene oxide]/[ethylene oxide] molar ratio of between 10/1 and 1/10.

24. The method of claim 23, further comprising, before reacting the polysaccharide with the modified polyetheramine:
activating at least one PS—COOH carboxylic function of the PS polysaccharide using a quaternary amine, and then adding said modified polyetheramine.

25. The method of claim 21, wherein all purification steps, after neutralization, are done at a temperature between 2° C. and 8° C.

26. The method of claim 21, further comprising, after (c), freeze-drying the product derived from (c), washing with the product derived from (c) ethanol, and drying the product derived from (c).

27. The method of claim 21, wherein said polysaccharide is selected in a group formed from pullulan, xanthan, alginate, and hyaluronic acid.

28. A modified polysaccharide obtained by the method of claim 21.

29. A hydrogel formed by at least one polysaccharide of claim 28 and an aqueous liquid, said aqueous liquid comprising serum and/or a cell culture medium.

30. The hydrogel of claim 29, wherein the hydrogel has heat-sensitive rheological properties with a transition temperature of between 33 and 39° or 4 and 20° C.
